(19) United States
(12) Patent Application Publication
(10) Pub. No.: US 2026/0098851 A1
(43) Pub. Date: Apr. 9, 2026

(54) OIL EVALUATION APPARATUS, OIL EVALUATION SYSTEM, INTERNAL COMBUSTION ENGINE-MOUNTED DEVICE, OIL EVALUATION METHOD, AND NON-TRANSITORY STORAGE MEDIUM

(71) Applicant: Isuzu Motors Limited, Yokohama-shi (JP)

(72) Inventors: Takuro MITA, Fujisawa-shi (JP); Junichiro NITTA, Fujisawa-shi (JP)

(73) Assignee: Isuzu Motors Limited, Yokohama-shi (JP)

(21) Appl. No.: 19/323,045

(22) Filed: Sep. 9, 2025

(30) Foreign Application Priority Data
Oct. 4, 2024 (JP) 2024-175298

(51) Int. Cl. G01N 33/28; G01N 9/00; G01N 27/06

(52) U.S. Cl. CPC G01N 33/2888; G01N 9/00; G01N 27/06; G01N 33/2847

(57) ABSTRACT

An oil evaluation apparatus includes an arithmetic process unit, and the arithmetic process unit is configured to calculate, based on a measurement result of a density of oil in an internal combustion engine, a mixing ratio of soot in the oil as an evaluation index.

### CROSS-REFERENCE TO RELATED APPLICATION

This application is based upon and claims the benefit of priority from prior Japanese Patent Application No. 2024-175298, filed Oct. 4, 2024, the entire contents of which are incorporated herein by reference.

### FIELD

The present invention relates generally to an oil evaluation apparatus, an oil evaluation system, an internal combustion engine-mounted device, an oil evaluation method, and a non-transitory storage medium.

### BACKGROUND

In an internal combustion engine of a vehicle that is an internal combustion engine-mounted device, soot occurs due to burning or the like of fuel and oil, and the soot that has occurred mixes in the oil. In addition, in the internal combustion engine, since the soot has a function of wearing a sliding part, an oil change of the oil that is used needs to be performed so that a mixing ratio of soot in oil may not exceed a reference level (refer to Jpn. Pat. Appln. KOKAI Publication No. 2005-43360). The time of the oil change is judged based on the driving time of the internal combustion engine, the travel time of the vehicle, an inspection of oil by visual observation, and the like.

### SUMMARY

According to an aspect of the present invention, an oil evaluation apparatus includes an arithmetic process unit, and the arithmetic process unit is configured to calculate, based on a measurement result of a density of oil in an internal combustion engine, a mixing ratio of soot in the oil as an evaluation index.

### BRIEF DESCRIPTION OF THE DRAWINGS

FIG. 1 is a schematic diagram illustrating an example of an oil evaluation system according to an embodiment.

FIG. 2 is a schematic diagram illustrating an example of a relationship of a mixing ratio of water in oil to an electrical resistance of the oil.

FIG. 3 is a flowchart schematically illustrating an example of a process of setting a reference density and a reference resistance value of oil, which is executed by an oil evaluation apparatus in the embodiment.

FIG. 4 is a flowchart schematically illustrating another example, different from the example of FIG. 3, of the process of setting the reference density and reference resistance value of oil, which is executed by the oil evaluation apparatus in the embodiment.

FIG. 5 is a flowchart schematically illustrating another example, different from the example of FIG. 3 and the example of FIG. 4, of the process of setting the reference density of oil, which is executed by the oil evaluation apparatus in the embodiment.

FIG. 6 is a flowchart schematically illustrating an example of an arithmetic process of a mixing ratio of soot in oil, which is executed by the oil evaluation apparatus in the embodiment.

FIG. 7 is a schematic diagram for describing an example of an arithmetic method of calculating the mixing ratio of soot in oil in the embodiment.

FIG. 8 is a schematic diagram illustrating an example of a system in which an evaluation result relating to the oil of the internal combustion engine by the oil evaluation apparatus in the embodiment is utilized.

### DETAILED DESCRIPTION

Hereinafter, an embodiment is described with reference to the accompanying drawings.

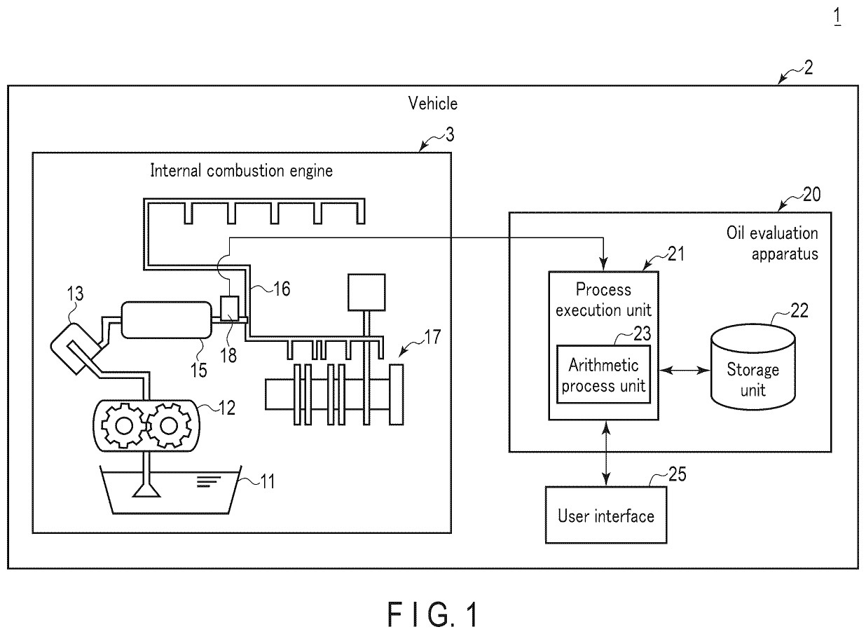

FIG. 1 is a schematic diagram illustrating an example of an oil evaluation system 1 according to the embodiment. As illustrated in FIG. 1, the oil evaluation system 1 includes a vehicle 2. The vehicle 2 includes an internal combustion engine 3. The internal combustion engine 3 is, for example, a diesel engine, and may be a gasoline engine. In the internal combustion engine 3, motive power is generated by burning fuel such as diesel oil or gasoline. In addition, in the internal combustion engine 3, oil (engine oil) is used as lubricating oil or the like. Note that in the description below, as an internal combustion engine-amounted device in which the internal combustion engine 3 is mounted, the vehicle 2 is described by way of example. However, processes and the like to be described below are applicable to cases where the internal combustion engine-amounted device is other than the vehicle 2. Examples of the internal combustion engine-amounted device other than the vehicle 2 include a stationary-type electric power generator, an industrial machine, and a motive power source apparatus.

As illustrated in FIG. 1, the internal combustion engine 3 includes an oil pan 11, an oil pump 12, an oil filter 13, and a heat exchanger 15. In the internal combustion engine 3, oil is stored in the oil pan 11, and the oil in the oil pan 11 is press-fed to each of supply targets by the oil pump 12. In the internal combustion engine 3, the oil is supplied to each of the supply targets through a supply path 16. In the internal combustion engine 3, for example, a piston crank mechanism 17 is provided as a supply target of oil. In the supply path 16, the oil filter 13 and the heat exchanger 15 are disposed between the oil pump 12 and the supply target, and, in the example of FIG. 1, the oil filter 13 and the heat exchanger 15 are disposed in the named order from a side (upstream side) near the oil pan 11. The oil filter 13 filters the oil, and the heat exchanger 15 cools the oil.

In the oil evaluation system 1, a measuring device 18 is mounted in the vehicle 2, and the measuring device 18 measures a density and electrical resistance of oil in the internal combustion engine 3. In one example, the measuring device 18 measures a temperature of oil in the internal combustion engine 3, in addition to the density and the electrical resistance of oil in the internal combustion engine 3. As the measuring device 18, devices that individually measure parameters one by one may be provided, or one device may measure all parameters. In a preferable example including the example of FIG. 1, the density and electrical resistance of oil are measured at a downstream-side position with respect to the oil pump 12 in the supply path 16, and the measurement is performed at a position between the oil pump 12 and the supply target. In the example of FIG. 1, the measurement by the measuring device 18 is performed at a position between the heat exchanger 15 and the supply target in the supply path 16. Note that in one example, the measurement by the measuring device 18 may be performed either at a position between the oil pump 12 and the oil filter 13, or at a position between the oil filter 13 and the heat exchanger 15. In addition, in another example, the measurement may be performed at an upstream-side position with respect to the oil pump 12, and the measurement may be performed, for example, in the oil pan 11.

The oil evaluation system 1 includes an oil evaluation apparatus 20. The oil evaluation apparatus 20 includes a process execution unit 21 and a storage unit 22, and the process execution unit 21 includes an arithmetic process unit 23. The storage unit 22 stores a program or the like executed by the process execution unit 21, and the arithmetic process unit 23 executes at least a part of the process by the process execution unit 21. The process execution unit 21 of the oil evaluation apparatus 20 performs a process of evaluation in regard to the oil in the internal combustion engine 3. At this time, the process execution unit 21 performs the process by executing an oil evaluation program stored in the storage unit 22 or the like.

In the example of FIG. 1, the oil evaluation apparatus 20 is mounted in the vehicle, and is composed of a vehicle-mounted computer such as a vehicle-mounted server. In this case, the process execution unit 21 is composed of a processor, an integrated circuit or the like of the vehicle-mounted computer, and the processor or the like, which constitutes the process execution unit 21, includes any one of an ECU (Electronic Control Unit), a CPU (Central Processing Unit), an ASIC (Application Specific Integrated Circuit), a microcomputer, an FPGA (Field Programmable Gate Array), and a DSP (Digital Signal Processor). The process execution unit 21 may be composed of a single processor or the like, or may be composed of a plurality of processors or the like. Besides, the storage unit 22 is composed of a storage medium (non-transitory storage medium), and includes either a main storage medium including a memory, or an auxiliary storage medium.

Note that in one example, a processor or the like of a computer on the outside of the vehicle 2 executes at least a part of the process by the process execution unit 21. In this case, the oil evaluation apparatus 20 is composed of one or more computers including the computer on the outside of the vehicle 2. In addition, in one example, the oil evaluation apparatus 20 is composed of a plurality of computers such as a plurality of servers, and the processors of the computers cooperate to execute a process to be described later by the process execution unit 21. In this case, for example, at least one vehicle-mounted computer and at least one computer on the outside of the vehicle 2 cooperate to execute a process that is based on the oil evaluation program.

Additionally, in one example, at least a part of the oil evaluation apparatus 20 is composed of a cloud server of a cloud environment. The infrastructure of the cloud environment is constituted by a virtual processor such as a virtual CPU, and a cloud memory. In a case where at least a part of the oil evaluation apparatus 20 is composed of the cloud server, the virtual processor executes at least a part of the process to be described later, which is based on the oil evaluation program. In this case, a program or the like, which is executed by the virtual processor, may be stored in the cloud memory.

In one example, the oil evaluation apparatus 20 is composed of either a vehicle-mounted computer or a computer on the outside of the vehicle 2, and the program executed by the process execution unit 21 is stored in a computer (server) connected to the oil evaluation apparatus 20 via a network, or in a cloud server or the like. In this case, the process execution unit 21 downloads programs including the oil evaluation program via a network. In addition, in one example, the oil evaluation apparatus 20 can communicate with either another computer or a cloud server by wireless communication or wired communication.

As illustrated in FIG. 1, the oil evaluation system 1 is provided with a user interface 25. In the example of FIG. 1, the user interface 25 is composed of a device mounted in the vehicle 2. However, in one example, at least a part of the user interface 25 may be composed of a device on the outside of the vehicle 2. In the user interface 25, various operations including operations relating to the evaluation of oil are input by a user or the like of the oil evaluation apparatus 20. Thus, the user interface 25 includes, as an operation input unit, one of a remote controller, a button, a mouse, a touch panel, and a keyboard. In addition, the user interface 25 is provided with a notification unit that notifies various kinds of information, and in the notification unit, information is notified by one of screen display, sound output, and the like. In one example, information indicating an evaluation result relating to the oil of the internal combustion engine 3 is notified in the notification unit.

In the internal combustion engine 3 of the vehicle 2, soot occurs due to burning or the like of fuel and oil, and the soot mixes in the oil. In addition, in the internal combustion engine 3, since the soot has a function of wearing a sliding part, an oil change of the oil that is used needs to be performed so that the mixing ratio of soot in oil may not exceed a reference level. Here, while the density of oil in an unused state is about 0.80 g/cm³ to 0.87 g/cm³ in a range of room temperatures, the density of soot is about 1.8 g/cm³. Thus, if the mixing ratio of soot in oil increases, the density of the oil increases.

In addition, in the internal combustion engine 3, in addition to soot, water and fuel mix in the oil. The density of water is about 1 g/cm³, and is higher than the density of oil. Thus, if the mixing ratio of water in the oil increases, the density of the oil increases. On the other hand, in the diesel engine that serves as the internal combustion engine 3, the density of diesel oil that is fuel is about 0.76 g/cm³ to 0.88 g/cm³, and is a value close to the density of oil. In addition, in the internal combustion engine 3, the mixing ratio of fuel in oil is normally less than 1%. Thus, a density variation of oil due to mixing of fuel in the oil is small.

From the above, by the use of oil in the internal combustion engine 3, the density of the oil varies due to the mixing of soot and water in the oil. Thus, in a case where the density of oil varies from a reference density ρobase to a density ρo, an element Δρo indicating a density variation of oil is divided into an element Δρbys indicating a density variation of oil due to mixing of soot, and an element Δρbyw indicating a density variation of oil due to mixing of water, and a relationship of an equation (1) is established. In addition, using the reference density ρobase of oil, a density ρs of soot, a mixing ratio Ws of soot, a density of ρw of water, and a mixing ratio Ww of water, the element Δρo becomes ρo-ρobase, the element Δρbys becomes (ρs−ρobase)×Ws, and the element Δρbyw becomes ((ρw−ρobase)×Ww. Thus, a relationship of an equation (2) is established.

$$ρ o - ρ obase = Δ ρ o = Δ ρ bys + Δ ρ byw \qquad ( 1 )$$

$$ρ o - ρ obase = ( ρ s - ρ obase ) \times Ws + ( pw - ρ obase ) \times Ww \qquad ( 2 )$$

In addition, while a volume resistivity of each of the oil and the diesel oil that is the fuel is about 1×10¹⁴ Ω·cm to 2×10¹⁴ Ω·cm, a volume resistivity of pure water is about 6×10⁵ Ω·cm and a volume resistivity of tap water is 5×10³ Ω·cm. Specifically, the volume resistivity of water is lower than the volume resistivity of each of the oil and the diesel oil. Thus, if the mixing ratio of water in oil increases, the electrical resistance of oil decreases. Note that a variation of electrical resistance of oil due to mixing of soot in the oil, and a variation of electrical resistance of oil due to mixing of fuel in the oil, are small. Although the soot has a high electrical conductivity, the soot mixed in the oil in the internal combustion engine 3 is dispersed. Thus, the influence of the mixed soot upon the electrical resistance of the oil is small.

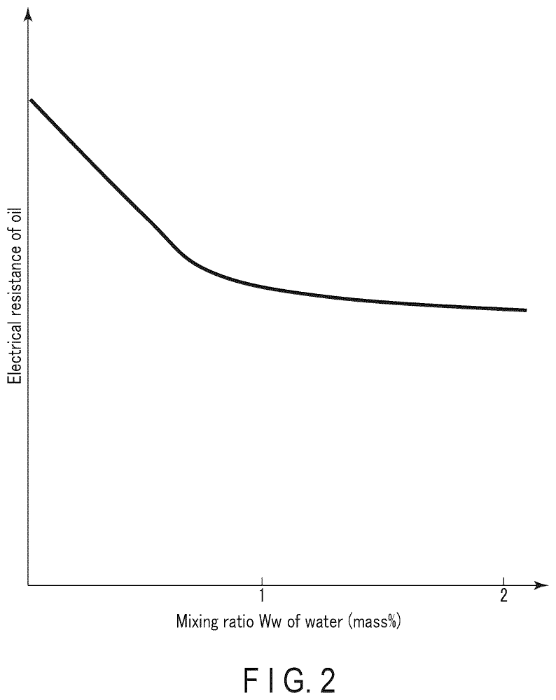

FIG. 2 is a schematic diagram illustrating an example of a relationship of the mixing ratio Ww of water in oil to the electrical resistance of the oil. FIG. 2 illustrates a graph, and the abscissa axis indicates the mixing ratio Ww of water by mass %, and the ordinate axis indicates the electrical resistance of the oil. In the example of FIG. 2, in a range in which the mixing ratio Ww is 1% or less, the electrical resistance of oil decreases in accordance with an increase of the mixing ratio Ww. In addition, in the internal combustion engine 3, the mixing ratio of water in oil is normally less than 18. Accordingly, by measuring the electrical resistance of oil in the internal combustion engine 3, the mixing ratio Ww of water can be derived based on the measurement result and the relationship of the mixing ratio Ww of water to the electrical resistance of oil.

In the present embodiment, the process execution unit 21 of the oil evaluation apparatus 20 executes the oil evaluation program, and thereby the arithmetic process unit 23 calculates, as an evaluation index, the mixing ratio of soot in oil in the internal combustion engine 3. At this time, the arithmetic process unit 23 calculates the mixing ratio of soot by utilizing the above-described characteristics of the oil, soot and water. The evaluation result relating to the oil includes a calculation result relating to the mixing ratio of soot. The arithmetic operation of the mixing ratio of soot is performed, for example, periodically at predetermined time intervals. Thereby, the time variation of the mixing ratio of soot is calculated in regard to the oil of the internal combustion engine 3. Hereinafter, a description is given of a process based on the oil evaluation program including an arithmetic process of the mixing ratio of soot in oil.

In the process based on the oil evaluation program, the arithmetic process unit 23 of the process execution unit 21 sets a reference for each of the density and the electrical resistance of oil of the internal combustion engine 3. Thereby, the reference density ρobase is determined in regard to the density of oil, and a reference resistance value is determined in regard to the electrical resistance of oil. At this time, the density of oil injected in the internal combustion engine 3 by an oil change or by simple oil replenishment without drawing-out of oil, or the density of oil in the internal combustion engine 3 in a state immediately after an oil change, is set as the reference density ρobase. In addition, the electrical resistance of oil injected in the internal combustion engine 3 in an oil change or in oil replenishment, or the electrical resistance of oil in the internal combustion engine 3 in a state immediately after an oil change, is set as the reference resistance value. Further, the arithmetic process unit 23 causes the storage unit 22 to store the set reference density ρobase and reference resistance value.

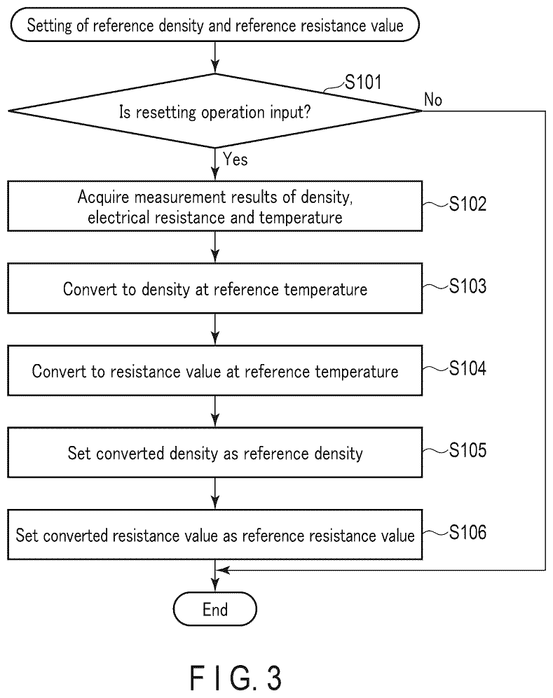

FIG. 3 is a flowchart schematically illustrating an example of a process of setting the reference density ρobase and the reference resistance value of oil, which is executed by the oil evaluation apparatus 20 in the embodiment. The process of the example of FIG. 3 is performed, for example, periodically at predetermined time intervals. If the process of the example of FIG. 3 is started, the arithmetic process unit 23 determines whether an operation of resetting the reference density ρobase and the reference resistance value of oil is input by the user interface 25 or the like (S101). If the resetting operation is not input (S101—No), the process of the example of FIG. 3 is ended. On the other hand, if the resetting operation is input (S101—Yes), the process execution unit 21 causes the measuring device 18 to measure the density, electrical resistance and temperature of oil in the internal combustion engine 3, and acquires measurement results of the density, electrical resistance and temperature of oil (S102).

Then, the arithmetic process unit 23 converts the measurement result of the density to a density at a reference temperature, based on the measurement result of the temperature (S103), and converts the measurement result of the electrical resistance to a resistance value at the reference temperature, based on the measurement result of the temperature (S104). In the conversion of the density, data indicating the relationship between the temperature and the density of oil, which is stored in the storage unit 22 or the like, is used, and in the conversion of the electrical resistance, data indicating the relationship between the temperature and the electrical resistance of oil, which is stored in the storage unit 22 or the like, is used. Then, the arithmetic process unit 23 sets the converted density as the reference density ρobase of oil (S105), and sets the converted resistance value as the reference resistance value of oil (S106).

In the case where the oil evaluation apparatus 20 executes the process of the example of FIG. 3, a user or the like of the oil evaluation apparatus 20 inputs the above-described resetting operation each time an oil change is performed. Thereby, each time an oil change is performed, the reference density ρobase and reference resistance value of oil are set (updated). At this time, with respect to the oil in the internal combustion engine 3 in the state immediately after the oil change, the density is set as the reference density ρobase, and the electrical resistance is set as the reference resistance value. Besides, in one example, in a case where simple oil replenishment without drawing-out of oil is performed, the above-described resetting operation may be input. In this case, with respect to the oil in the internal combustion engine 3 in the state immediately after the oil replenishment, the density can be set as the reference density ρobase, and the electrical resistance can be set as the reference resistance value.

Note that in the internal combustion engine 3, oil is used in a temperature range of −20° C. to 120° C., and immediately after a warm-up operation is performed, the temperature of oil in the internal combustion engine 3 falls within a range of 80° C. to 120° C. In one example, the measuring device 18 can measure the density and electrical resistance of oil in a temperature range of 60° C. to 100° C. In addition, as the reference temperature, one or more temperatures in a temperature range of 20° C. to 100° C. are set, and, for example, 20° C., 25° C., and 40° C. are set as reference temperatures.

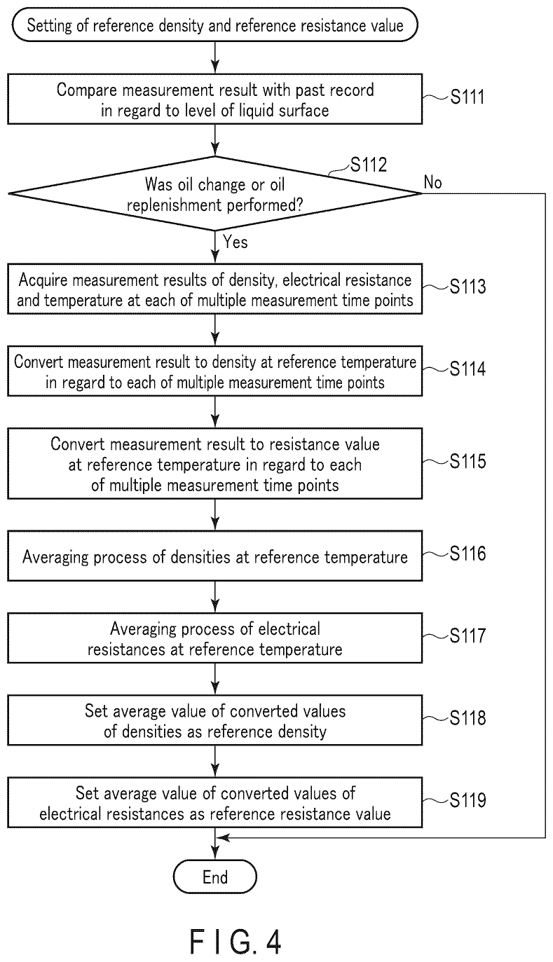

FIG. 4 is a flowchart schematically illustrating another example, different from the example of FIG. 3, of the process of setting the reference density ρobase and reference resistance value of oil, which is executed by the oil evaluation apparatus 20 in the embodiment. The process of the example of FIG. 4 is executed while the operation of the internal combustion engine 3 is stopped, or before the operation of the internal combustion engine 3 is started. In addition, in a case where the process of the example of FIG. 4 is executed, the internal combustion engine 3 is provided with an oil level sensor that detects a level (position) of a liquid surface of oil in the oil pan 11. Here, while the internal combustion engine 3 is operating, or immediately after the stop of the operation of the internal combustion engine 3, the liquid surface of oil is not stable. Thus, the process execution unit 21 or the like determines that the liquid surface of oil is stable, based on the fact that a time from the stop of the operation of the internal combustion engine 3 is a predetermined period or more, or based on the fact that a difference of the temperature of cooling water or lubricating oil of the internal combustion engine 3 from the temperature of the cooling water or lubricating oil at the time of the stop of the internal combustion engine 3 is sufficiently large. In addition, upon determining that the liquid surface of oil is stable, the process execution unit 21 or the like records the level of the liquid surface of oil.

If the process of the example of FIG. 4 is started, the arithmetic process unit 23 compares the level of the liquid surface of oil in real time, which is measured in the internal combustion engine 3, with the level of the liquid surface of oil recorded in the past. Specifically, the arithmetic process unit 23 compares the measurement result in real time with the past record in regard to the level of the liquid surface of oil (S111). At this time, the comparison is performed based on the detection result of the level of the liquid surface in the oil level sensor. In addition, based on at least the comparison result of the level of the liquid surface of oil, the arithmetic operation unit 23 determines whether the oil change or the simple oil replenishment without drawing-out of oil was performed (S112). At this time, the arithmetic process unit 23 calculates an increase amount of the liquid level of oil recorded in real time, relative to the liquid level of oil in the past recorded in the storage unit 22 of the internal combustion engine 3, and executes the determination, based on at least the calculated increase amount. The arithmetic process unit 23 determines that the oil change or the oil replenishment was performed, for example, based on the fact that the above-described increase amount is a reference increase amount or more.

Here, even in a case where the level of the liquid surface of the oil in the internal combustion engine 3 is a lower-limit value or thereabout, if the above-described increase amount is less than the reference increase amount, the arithmetic process unit 23 determines that this case is uncertain data, and determines neither that the oil change was performed, nor that the oil replenishment was performed. In addition, even in a case where the above-described increase amount is equal to or greater than the reference increase amount, if either the travel time of the vehicle 2 or the driving time of the internal combustion engine 3 from a previous oil change is shorter than a reference range, the arithmetic process unit 23 determines that this case is uncertain data, and determines neither that the oil change was performed, nor that the oil replenishment was performed.

In one example, in each of the preceding and previous oil changes, the arithmetic process unit 23 records in the storage unit 22 the set reference density ρobase and reference resistance value of oil. In addition, even in a case where the above-described increase amount is equal to or greater than the reference increase amount, if the difference of either the reference density ρobase or the reference resistance value of oil, which is derived by a process to be described later, from the value set in the past oil change exceeds a reference range, the arithmetic process unit 23 determines that this case is uncertain data, and determines neither that the oil change was performed, nor that the oil replenishment was performed. Besides, the mixing ratio of soot in oil becomes smaller after the oil change than before the oil change, and the density of oil varies. In one example, even in a case where the above-described increase amount is equal to or greater than the reference increase amount, if the variation of the density of oil before and after the stop of the internal combustion engine 3 is less than a reference range, the arithmetic process unit 23 determines that this case is uncertain data, and determines neither that the oil change was performed, nor that the oil replenishment was performed. Note that in another example, in a case where the above-described increase amount is equal to or greater than the reference increase amount, but the variation of the density of oil before and after the stop of the internal combustion engine 3 is less than a reference range, the arithmetic process unit 23 may determine that the oil change was not performed but the oil replenishment without drawing-out of oil was performed.

If it is not determined that either the oil change or the oil replenishment was performed (S112—No), the process of the example of FIG. 4 is ended. On the other hand, if it is determined that either the oil change or the oil replenishment was performed (S112—Yes), the process execution unit 21 causes the measuring device 18 to measure the density, electrical resistance and temperature of oil in the internal combustion engine 3 at each of multiple measurement time points, and acquires measurement results of the density, electrical resistance and temperature of oil at the multiple measurement time points (S113). At this time, for example, while the temperature of oil in the internal combustion engine 3 is varying with time, the measurement is performed at the multiple measurement time points. Note that while the measurement of the level of the liquid surface of oil is performed in the state in which the liquid surface is stable, such as in a state immediately before the start of the internal combustion engine 3, the measurement of the density, electrical resistance and temperature of oil is performed in the state in which the internal combustion engine 3 is operating after the start of the internal combustion engine 3. Thus, in S113, the measurement results of the density, electrical resistance and temperature of oil are acquired with respect to each of the multiple measurement time points while the internal combustion engine 3 is operating. In regard to each of the multiple measurement time points, based on the measurement result of the temperature, the arithmetic process unit 23 converts the measurement result of the density to a density at the reference temperature (S114), and converts the measurement result of the electrical resistance to a resistance value at the reference temperature (S115).

In addition, the arithmetic process unit 23 executes an averaging process of the densities at the reference temperature, which were calculated with respect to the multiple measurement time points (S116), and executes an averaging process of the electrical resistances at the reference temperature, which were calculated with respect to the multiple measurement time points (S117). Then, the arithmetic process unit 23 sets an average value of the converted values of the densities at the multiple measurement time points as the reference density ρobase of oil (S118), and sets an average value of the converted values of the electrical resistances at the multiple measurement time points as the reference resistance value of oil (S119). Thereby, in regard to the oil in the internal combustion engine 3 in the state immediately after the oil change, the density is set to the reference density ρobase, and the electrical resistance is set to the reference resistance value.

In another example different from the example of FIG. 3 and the example of FIG. 4, the user or the like of the oil evaluation apparatus 20 can input an operation of setting the reference density ρobase and the reference resistance value in the user interface 25. In this case, based on the operation input in the user interface 25, the arithmetic process unit 23 sets the reference density ρobase and the reference resistance value. In the present example, the kind or the like of the oil injected in the internal combustion engine 3 in the oil change is recognized by the user or the like of the oil evaluation apparatus 20, and the user or the like of the oil evaluation apparatus 20 inputs the operation in the user interface 25, based on the recognized kind or the like of the oil. In the present example, in regard to the oil injected in the internal combustion engine 3 in the oil change, the density is set to the reference density ρobase, and the electrical resistance is set to the reference resistance value.

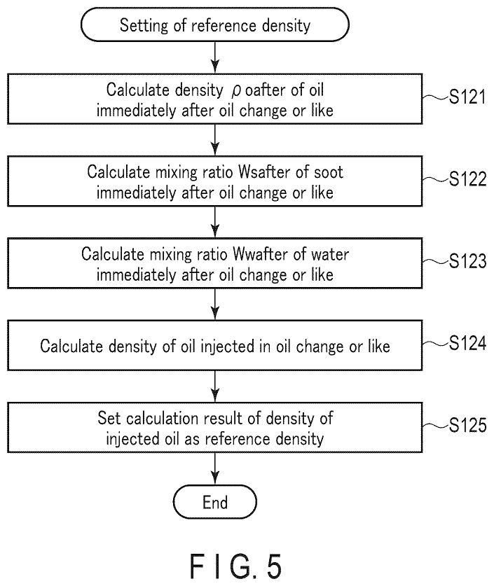

Besides, the reference density ρobase of oil can also be set by a method to be described below. FIG. 5 is a flowchart schematically illustrating another example, different from the example of FIG. 3 and the example of FIG. 4, of the process of setting the reference density ρobase of oil, which is executed by the oil evaluation apparatus 20 in the embodiment. The process of the example of FIG. 5 is executed, for example, each time either the oil change or the simple oil replenishment without drawing-out of oil is performed. If the process of the example of FIG. 5 is started, the arithmetic process unit 23 acquires measurement results of the density and temperature of oil in the internal combustion engine 3 in the state immediately after the oil change or the like (including oil replenishment). Then, the arithmetic process unit 23 calculates a density ρoafter of oil at the reference temperature immediately after the oil change or the like, like either the calculation of the reference density ρobase in the example of FIG. 3, or the calculation of the reference density ρobase in the example of FIG. 4 (S121).

Then, the arithmetic process unit 23 calculates a mixing ratio Wsafter of soot in oil in the internal combustion engine 3 in the state immediately after the oil change or the like (including oil replenishment) (S122). Here, even if the oil is drawn out from the internal combustion engine 3 in the oil change, oil remains in the oil filter 13 or the like. Thus, even in the state immediately after the oil change, soot mixes in the oil. In addition, there is a case where simple oil replenishment is performed to replenish unused oil without drawing out the oil from the internal combustion engine 3. Using a volume Vres of residual oil in the oil change, a volume Vadd of oil injected in the oil change, and a mixing ratio Wsbefore of soot in oil immediately before the oil change, the arithmetic process unit 23 calculates the mixing ratio Wsafter of soot, as expressed in equation (3).

$$Wsafter = Wsbefore \times \frac{Vres}{Vadd + Vres} \qquad ( 3 )$$

Here, in a case where it is determined that the oil change was performed, the volume Vres of the residual oil is calculated by using a value that can be specified by specifications or the like of the internal combustion engine 3. In addition, in a case where it is determined that replenishment of unused oil without drawing-out of oil (simple oil replenishment) was performed, the volume Vres of the residual oil is calculated by using the level of the liquid surface by the oil level sensor before the oil replenishment, and the volumes and shapes of the internal combustion engine 3 and fittings to the internal combustion engine 3. The arithmetic process unit 23 stores the calculated volume Vres in the storage unit 22. The volume Vadd of the injected oil can be recognized by a worker that performed the oil change. The user or the like of the oil evaluation apparatus 20 can input, in the user interface 25, information on whether the oil change was performed or the oil replenishment without drawing-out of oil was performed, and the volume Vadd of the injected oil. In addition, the volume Vadd can be calculated by using the detection result of the level of the liquid surface in the oil level sensor, the volumes and shapes of the internal combustion engine 3 and fittings to the internal combustion engine 3, and the above-described volume Vres. Furthermore, the mixing ratio Wsbefore of soot immediately before the oil change can be calculated by deriving the mixing ratio Ws of soot by a process to be described later, immediately before the oil change or the oil replenishment without drawing-out of oil.

The arithmetic process unit 23 calculates a mixing ratio Wwafter of water in oil in the internal combustion engine 3 in the state immediately after the oil change or the like (including oil replenishment) (S123). In one example, the arithmetic process unit 23 causes the electrical resistance of oil to be measured immediately after the oil change or the like, and calculates the mixing ratio Wwafter of water in oil, based on the measurement result of the electrical resistance. At this time, the mixing ratio Wwafter of water immediately after the oil change or the like is calculated, in the same manner as in the arithmetic process (to be described later) of calculating the mixing ratio Ww of water in oil, based on the electrical resistance of oil. In another example, like the calculation of the mixing ratio Wsafter, the mixing ratio Wwafter of water in oil immediately after the oil change or the like is calculated by using the volume Vres of residual oil in the oil change or the like, the volume Vadd of oil injected in the oil change or the like, and the mixing ratio Wwbefore of water in oil immediately before the oil change. Note that the mixing ratio Wwbefore of water immediately before the oil change or the like can be calculated by deriving the mixing ratio Ww of water by the process to be described later.

In addition, the arithmetic process unit 23 calculates the density of oil injected in the internal combustion engine 3 in the oil change or the like (including oil replenishment), by using the density ρoafter of oil immediately after the oil change or the like, the mixing ratio Wsafter of soot immediately after the oil change or the like, and the mixing ratio Wwafter of water immediately after the oil change or the like (S124). Then, the arithmetic process unit 23 sets the calculation result of the density of the injected oil as the reference density ρobase of oil (S125). Here, if the above-described equation (2) is modified, the reference density ρobase of oil is derived as expressed in equation (4). In the process of S124, in equation (4), the density ρoafter of oil immediately after the oil change or the like is substituted for the density ρo, the mixing ratio Wsafter of soot immediately after the oil change or the like is substituted for the mixing ratio Ws, and the mixing ratio Wwafter of water immediately after the oil change or the like is substituted for the mixing ratio Ww. In addition, as the reference density ρobase, the density of the injected oil is calculated as expressed in equation (5).

$$ρ obase = \frac{ρ o - Ws \times ρ s - Ww \times ρ w}{1 - Ws - Ww} \qquad ( 4 )$$

$$ρ obase = \frac{ρ oafter - Wsafter \times ρ s - Wwafter \times ρ w}{1 - Wsafter - Wwafter} \qquad ( 5 )$$

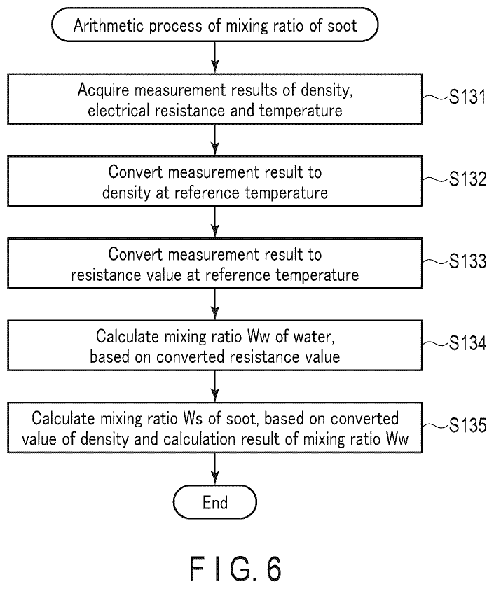

By executing the process based on the oil evaluation program, the arithmetic process unit 23 executes the arithmetic process of the mixing ratio of soot in oil, in the state in which the reference density ρobase and the reference resistance value are set in the same manner as in one of the above-described examples. FIG. 6 is a flowchart schematically illustrating an example of the arithmetic process of the mixing ratio Ws of soot in oil, which is executed by the oil evaluation apparatus 20 in the embodiment. The process of the example of FIG. 6 is executed, for example, periodically at predetermined time intervals, after setting the reference density ρobase and the reference resistance value of oil immediately after the oil change.

If the process of the example of FIG. 6 is started, the arithmetic process unit 23 causes the measuring device 18 to measure the density, electrical resistance and temperature of oil, and acquires measurement results in real time in regard to the density, electrical resistance and temperature of oil (S131). Then, based on the measurement result of the temperature, the arithmetic process unit 23 converts the measurement result of the density to a density at the reference temperature (S132), and converts the measurement result of the electrical resistance to a resistance value at the reference temperature (S133).

Then, based on the converted resistance value, the arithmetic process unit 23 calculates the mixing ratio Ww of water in oil in real time (S134). At this time, the mixing ratio Ww of water in real time is calculated by using the data indicating the relationship of the mixing ratio Ww of water to the electrical resistance of oil at the reference temperature, which is stored in the storage unit 22 or the like, in addition to the converted value of the electrical resistance at the reference temperature. In the data indicating the relationship of the mixing ratio Ww of water to the electrical resistance of oil, a table or an approximate expression for converting the electrical resistance of oil to the mixing ratio Ww is indicated, or, in one example, the relationship of the mixing ratio Ww of water to the electrical resistance of oil, which is illustrated in the example of FIG. 2, is indicated.

Furthermore, the arithmetic process unit 23 calculates the mixing ratio Ws of soot in oil in real time, based on the converted value of the density of oil at the reference temperature, and the calculation result of the mixing ratio Ww of water (S135). Thereby, the mixing ratio Ws of soot in oil is calculated based on the measurement result of the density ρo of oil, and the calculation result of the mixing ratio Ww of water in oil. Here, if the above-described equation (2) is modified, the mixing ratio Ws of soot is derived as expressed in equation (6). In the process of S135, the arithmetic process unit 23 substitutes appropriate values for the reference density ρobase and density ρo of oil, the density ρw of water, the density ρs of soot, and the mixing ratio Ww of water in equation (6). At this time, as the density ρo, the converted value obtained by converting the measurement result to the density at the reference temperature is substituted, and, as the densities ρw and ρs, the values stored in the storage unit 22 or the like are substituted. In addition, as the mixing ratio Ww of water, the calculation result in S134 is substituted.

$$Ws = \frac{ρ s - ρ obase - ( ρ w - ρ obase ) \times Ww}{ρ s - ρ obase} \qquad ( 6 )$$

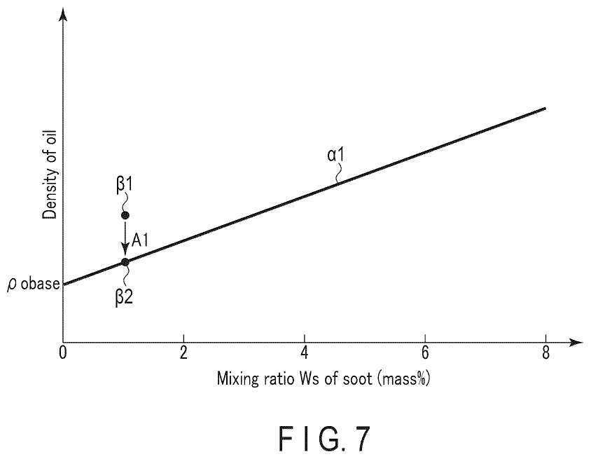

FIG. 7 is a schematic diagram for describing an example of an arithmetic method of calculating the mixing ratio Ws of soot in oil in the embodiment. FIG. 7 illustrates a graph in which the abscissa axis indicates the mixing ratio Ws of soot, and the ordinate axis indicates the density ρo of oil, and the mixing ratio Ws of soot is indicated by mass %. In addition, in FIG. 7, a line al indicates the relationship of the density ρo of oil to the mixing ratio Ws of soot in a case where a substance other than soot, such as water, is not mixed in oil. By the line al, the variation of the density of oil from the reference density ρobase in relation to the mixing ratio Ws of soot is indicated in regard to the case where a substance other than soot is not mixed in oil.

In the present embodiment, the mixing ratio Ws of soot is calculated by the above-described equation (6). Thus, based on the measurement result of the density of oil, the element Δρo (=ρo−ρobase) indicating the density variation of oil is calculated, and, based on the calculation result of the mixing ratio Ww of water, the element Δρbyw (=(ρw−ρobase)×Ww) indicating the density variation of oil due to mixing of water is calculated. In addition, the mixing ratio Ws of soot is calculated by excluding the element Δρbyw indicating the density variation of oil due to mixing of water from the element Δρo indicating the density variation of oil.

By excluding the element Δρbyw indicating the density variation due to mixing of water, in the example of FIG. 7, a data point β1 corresponding to the measurement result of the density moves to a data point β2 on the line α1 (arrow A1). In addition, the mixing ratio Ws of soot is calculated in accordance with the density at the data point β2 and the relationship of the density ρo of oil to the mixing ratio Ws of soot indicated by the line α1.

In addition, in one example, the arithmetic process unit 23 calculates the mixing ratio Ws of soot, based on the measurement accuracy or the like of the electrical resistance and density of oil, in addition to the calculation result of the mixing ratio Ww of water and the measurement result of the density of oil. Here, in the case of calculating the mixing ratio Ww of water as described above, the measurement accuracy in the measuring device 18 affects the calculation result. In addition, in a case where the size of soot particles mixed in oil is greater than a reference range, the density of soot becomes greater than a reference range, and the calculation result of the mixing ratio Ws of soot is affected.

Thus, in the present example, in addition to the reference density ρobase and density ρo of oil, the density of ρw of water, the density of ρs of soot and the mixing ratio Ww of water, a coefficient ks relating to the density variation of oil due to mixing of soot, and a coefficient kw relating to the density variation of oil due to mixing of water are used for the arithmetic operation of the mixing ratio Ws of soot. If the coefficients ks and kw are defined as described above, the element Δρo indicating the density variation of oil has a relationship of equation (7) that is established in relation to the element Δρbys indicating the density variation of oil due to mixing of soot, and the element Δρbyw indicating the density variation of oil due to mixing of water. In addition, if equation (7) is modified like the modification of equation (1) to equation (2), the relationship of equation (8) is established.

$$Δρ o = ks \times Δ ρ bys + kw \times Δ ρ byw \qquad ( 7 )$$

$$ρ o - ρ obase = ks \times ( ρ s - ρ obase ) \times Ws + kw \times ( pw - ρ obase ) \times Ww \qquad ( 8 )$$

In addition, if the above-described equation (8) is modified, the mixing ratio Ws of soot is derived as expressed in equation (9). In the present example, the arithmetic process unit 23 calculates the mixing ratio Ws of soot, by substituting appropriate values for the reference density ρobase and density ρo of oil, the density of ρw of water, the density of ρs of soot, the mixing ratio Ww of water, and the coefficient ks and kw in equation (9). At this time, similar values to the arithmetic operation of the mixing ratio Ww by equation (6) are substituted for the densities po, ρw and ρs and the mixing ratio Ww. The value of the coefficient ks is adjusted to an appropriate value in accordance with the measurement accuracy of density, and the size or the like of mixed soot. For example, in a case where the size of mixed soot particles is greater than a reference range, the coefficient ks is adjusted to a smaller value than in the case where the size of soot particles is within the reference range. In addition, the value of the coefficient kw is adjusted to an appropriate value in accordance with the measurement accuracy or the like of the electrical resistance.

$$Ws = \frac{ρ s - ρ obase - kw \times ( ρ w - ρ obase ) \times Ww}{ks \times ( ρ s - ρ obase )} \qquad ( 9 )$$

In the oil evaluation apparatus 20, the process execution unit 21 may notify the mixing ratio Ws of soot calculated as the evaluation index as described above, by using the user interface 25. In this case, for example, the calculation result of the mixing ratio Ws of soot is notified by one of screen display, sound output, and the like. Besides, the process execution unit 21 may transmit the evaluation result of oil including the calculation result of the mixing ratio Ws of soot, to a computer (server) or a cloud server, which is other than the oil evaluation apparatus 20.

As described above, in the embodiment and the like, based on the measurement result of the density of oil in the internal combustion engine 3, the mixing ratio Ws of soot in oil is calculated as the evaluation index. The density of soot is higher than the density of oil. Thus, based on the time variation or the like of oil until reaching the density that is the measurement result, the mixing ratio Ws of soot can appropriately be derived. In addition, in the embodiment and the like, the mixing ratio Ww of water in oil is calculated based on the measurement result of the electrical resistance of oil in the internal combustion engine 3, and the mixing ratio Ws of soot is calculated based on the measurement result of the density of oil, and the calculation result of the mixing ratio Ww of water. Accordingly, the mixing ratio Ws of soot is appropriately derived by also taking into account the mixing of water in oil.

Additionally, in the case of calculating the mixing ratio Ws of soot by using one of equations (6) and (9), the element Δρo indicating the density variation of oil is calculated based on the measurement result of the density of oil, and the element Δρbyw indicating the density variation of oil due to mixing of water is calculated based on the calculation result of the mixing ratio Ww of water. In addition, the mixing ratio Ws of soot is calculated by excluding the element Δρbyw indicating the density variation of oil due to mixing of water from the element Δρ indicating the density variation of oil. Thus, even in the state in which water mixes in oil, the mixing ratio Ws of soot in oil can appropriately be derived.

Additionally, in one example of the embodiment and the like, the measurement result of the electrical resistance of oil is converted to the resistance value at the reference temperature, based on the measurement result of the temperature of oil in the internal combustion engine 3, and the mixing ratio Ww of water is calculated based on the converted resistance value. In addition, the measurement result of the density of oil is converted to the density at the reference temperature, based on the measurement result of the temperature of oil, and the mixing ratio Ws of soot is calculated based on the converted density. Thus, even in the case of measuring the electrical resistance and density of oil under the circumstance in which the temperature of oil varies with time, the mixing ratio Ww of water and the mixing ratio Ws of soot are appropriately calculated. In addition, in the case of calculating the mixing ratio Ws of soot by using equation (9), the mixing ratio Ws of soot is calculated based on the measurement accuracies or the like of the electrical resistance and density of oil, in addition to the calculation result of the mixing ratio Ww of water and the measurement result of the density of oil. Thus, the mixing ratio Ws of soot can more appropriately be derived.

Additionally, in one example of the embodiment and the like, the density of oil injected in the internal combustion engine 3 in the oil change, or the density of oil in the internal combustion engine 3 in the state immediately after the oil change, is set as the reference density ρobase. In addition, the mixing ratio Ws of soot in oil is calculated based on the density variation of the measurement result of the density of oil in relation to the reference density ρobase. Thus, the density of oil in the state in which soot is not mixed or soot is scarcely mixed is appropriately set as the reference density ρobase. In addition, since the mixing ratio Ws of soot is calculated based on the variation of the density of oil from the appropriately set reference density ρobase, the mixing ratio Ws is further appropriately derived.

Additionally, in one preferable example of the embodiment, the density and electrical resistance of oil are measured at a downstream-side position with respect to the oil pump 12 in the supply path 16 of oil of the internal combustion engine 3, that is, at a position between the oil pump 12 and the supply target. Here, in the oil pan 11, air flows into the stored oil, the air as bubbles is taken in the oil, and the bubbles deteriorate the measurement accuracy of the density and electrical resistance of oil. In the present example, since the measurement is performed between the oil pump 12 and the supply target in which pressure rises, the density and electrical resistance of oil are measured by being hardly affected by the bubbles of air. Thereby, the density and electrical resistance of oil are appropriately measured, and the mixing ratio Ws of soot in oil is further appropriately derived.

As described above, in the embodiment and the like, the mixing ratio Ws of soot in oil is appropriately derived. Thus, based on the derived mixing ratio Ws of soot, the user or the like of the oil evaluation apparatus 20 can appropriately judge whether or not to perform the oil change of the internal combustion engine 3.

Accordingly, based on the derived result of the mixing ratio Ws of soot, the oil change can be performed at an appropriate time.

Note that, for example, in a case where the electrical resistance and density of oil are measured only at the reference temperature and thereabout, the process of converting the measurement results to the density and resistance value at the reference temperature may not necessarily be executed. In addition, for example, in a case where it is clear in advance that water is scarcely mixed in oil, the measurement of the electrical resistance of oil and the arithmetic process of the mixing ratio Ww of water may not necessarily be executed. In this case, for example, assuming that the mixing ratio Ww of water is 0%, the arithmetic process unit 23 calculates the mixing ratio Ws of soot in oil, based on the measurement result of the density of oil.

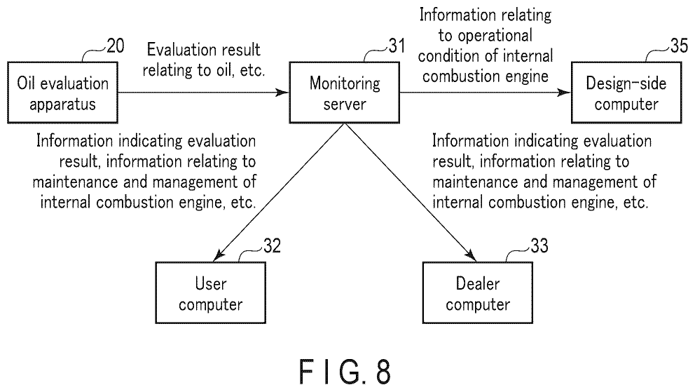

FIG. 8 is a schematic diagram illustrating an example of a system in which the evaluation result relating to the oil of the internal combustion engine 3 by the oil evaluation apparatus 20 in the embodiment is utilized. The system of the example of FIG. 8 includes, in addition to the oil evaluation apparatus 20, a monitoring server 31, a user computer 32, a dealer computer 33 and a design-side computer 35. The monitoring server 31 is composed of a computer or a cloud server, which is other than the oil evaluation apparatus 20. The monitoring server 31 performs monitoring relating to internal combustion engines in regard to vehicles including the vehicle 2, which are distributed on the market.

The oil evaluation apparatus 20 transmits the evaluation result relating to the oil of the internal combustion engine 3, which includes the mixing ratio Ws of soot calculated as the evaluation index, to the monitoring server 31. Each time the oil evaluation apparatus 20 executes the arithmetic process of the mixing ratio Ws of soot, the oil evaluation apparatus 20 transmits the derived result of the mixing ratio Ws of soot to the monitoring server 31. Thus, the monitoring server 31 acquires the time variation of the mixing ratio Ws of soot in oil in regard to the internal combustion engine 3 of the vehicle 2. Based on the time variation or the like of the mixing ratio Ws of soot, the monitoring server 31 may estimate a time of a change of oil in the internal combustion engine 3 of the vehicle 2. In this case, the information relating to the maintenance and management of the internal combustion engine 3, which includes the time of the change of oil, is generated.

Note that in one example, the monitoring server 31 may perform at least a part of the above-described process by the oil evaluation apparatus 20. In this case, for example, the measurement results of the density and electrical resistance of oil in the internal combustion engine 3, the level of the liquid surface of oil, and the information of the user interface or the like, are transmitted to the monitoring server 31 that serves as the oil evaluation apparatus 20. In addition, the monitoring server 31 derives the mixing ratio Ww of water in oil and the mixing ratio Ws of soot in oil, in the above-described manner.

The user computer 32 is composed of a terminal, a server or the like used by the user of the vehicle 2. In addition, the dealer computer 33 is composed of a terminal, a server or the like used by a dealer of vehicles including the vehicle 2, which are distributed on the market. The monitoring server 31 transmits either the information indicating the evaluation result of the oil of the internal combustion engine 3, or the information relating to the maintenance and management of the internal combustion engine 3 of the vehicle 2, to each of the user computer 32 and the dealer computer 33. Based on the information that the user computer 32 receives from the monitoring server 31, the user of the vehicle 2 asks the dealer for entrance of the vehicle 2 in the garage, conducts consultation about the vehicle 2 to the dealer, and provides data relating to the vehicle 2 to the dealer. On the other hand, based on the information that the dealer computer 33 receives from the monitoring server 31, the dealer provides, for the user of the vehicle 2, the support and guaranteeing relating to the vehicle 2, proposals for an oil change and parts replacement and the like, and plans relating to the vehicle 2.

In addition, in the example of FIG. 8, based on the time variation of the mixing ratio Ws of soot, and the like, the monitoring server 31 generates the information relating to the operational condition of the internal combustion engine 3. In the information relating to the operational condition of the internal combustion engine 3, how the internal combustion engine 3 has been driven, or the like, is indicated. The design-side computer 35 is composed of a terminal, a server or the like used by a designer of vehicles including the vehicle 2, which are distributed on the market, and, in particular, is composed of a terminal, a server or the like used by a designer of internal combustion engines of vehicles that are distributed on the market. The monitoring server 31 transmits the information relating to the operational condition of the internal combustion engine 3 to the design-side computer 35. Thereby, the operational condition of the internal combustion engine 3 is fed back to the designer of the internal combustion engine 3. Based on the information from the monitoring server 31, the designer of the internal combustion engine 3 performs supports on the market on which vehicles are distributed. In addition, based on the information from the monitoring server 31, the designer updates devices, products and the like including software, which are used in internal combustion engines and vehicles.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An oil evaluation apparatus comprising an arithmetic process unit configured to calculate, based on a measurement result of a density of oil in an internal combustion engine, a mixing ratio of soot in the oil as an evaluation index.

2. The oil evaluation apparatus of claim 1, wherein
the arithmetic process unit is configured to calculate a mixing ratio of water in the oil, based on a measurement result of electrical resistance of the oil in the internal combustion engine, and
the arithmetic process unit is configured to calculate the mixing ratio of the soot in the oil, based on a calculation result of the mixing ratio of the water, in addition to the measurement result of the density of the oil.

3. The oil evaluation apparatus of claim 2, wherein in an arithmetic operation of the mixing ratio of the soot, the arithmetic process unit is configured to:
calculate an element indicating a density variation of the oil, based on the measurement result of the density of the oil;
calculate an element indicating a density variation of the oil due to mixing of water, based on the calculation result of the mixing ratio of the water; and
calculate the mixing ratio of the soot, by excluding the element indicating the density variation of the oil due to the mixing of the water from the element indicating the density variation of the oil.

4. The oil evaluation apparatus of claim 2, wherein in an arithmetic operation of the mixing ratio of the water, the arithmetic process unit is configured to convert the measurement result of the electrical resistance of the oil to a resistance value at a reference temperature, based on a measurement result of a temperature of the oil in the internal combustion engine, and configured to calculate the mixing ratio of the water, based on the converted resistance value.

5. The oil evaluation apparatus of claim 2, wherein in an arithmetic operation of the mixing ratio of the soot, the arithmetic process unit is configured to calculate the mixing ratio of the soot, based on measurement accuracies of the electrical resistance and the density of the oil, in addition to the calculation result of the mixing ratio of the water and the measurement result of the density of the oil.

6. The oil evaluation apparatus of claim 1, wherein in an arithmetic operation of the mixing ratio of the soot, the arithmetic process unit is configured to convert the measurement result of the density of the oil to a density at a reference temperature, based on a measurement result of a temperature of the oil, and configured to calculate the mixing ratio of the soot, based on the converted density.

7. The oil evaluation apparatus of claim 1, wherein
the arithmetic process unit is configured to set, as a reference density, a density of oil injected in the internal combustion engine, or a density of oil in the internal combustion engine in a state immediately after an oil change, and
in an arithmetic operation of the mixing ratio of the soot, the arithmetic process unit is configured to calculate the mixing ratio of the soot in the oil, based on a density variation of the measurement result of the density of the oil in relation to the reference density.

8. An oil evaluation system comprising:
the oil evaluation apparatus of claim 1;
a vehicle including the internal combustion engine, the mixing ratio of the soot in the oil in the internal combustion engine of the vehicle being calculated by the oil evaluation apparatus; and
a measuring device configured to measure the density of the oil in the internal combustion engine.

9. An internal combustion engine-mounted device comprising:
the oil evaluation apparatus of claim 1;
the internal combustion engine in which the mixing ratio of the soot in the oil that is used is calculated by the oil evaluation apparatus; and
a measuring device configured to measure the density of the oil in the internal combustion engine.

10. An oil evaluation method comprising calculating, based on a measurement result of a density of oil in an internal combustion engine, a mixing ratio of soot in the oil as an evaluation index.

11. A non-transitory storage medium storing an oil evaluation program, the oil evaluation program causing a computer to calculate, based on a measurement result of a density of oil in an internal combustion engine, a mixing ratio of soot in the oil as an evaluation index.
